(19) United States
(12) Patent Application Publication
(10) Pub. No.: US 2007/0066548 A1
(43) Pub. Date: Mar. 22, 2007

(54) Globin variant gene methods and compositions

(75) Inventor: Ronald L. Nagel, New York, NY (US)

Correspondence Address: AMSTER, ROTHSTEIN & EBENSTEIN LLP, 90 PARK AVENUE, NEW YORK, NY, 10016, US

(73) Assignee: ALBERT EINSTEIN COLLEGE OF MEDICINE OF YESHIVA UNIVERSITY, BRONIX, NEW YORK, NY (US)

(21) Appl. No.: 10/547,980

(22) PCT Filed: Mar. 12, 2004
(86) PCT No.: PCT/US04/07325

§ 371 Date: Sep. 15, 2006

Related U.S. Application Data
(60) Provisional application No. 60/454,808, filed on Mar. 14, 2003.

(51) Int. Cl. A61K 48/00

(52) U.S. Cl. 514/44

(57) ABSTRACT

Methods are provided for treating a mammal having a disease, where the disease is caused by a globin disease mutation or a mutation in globin regulation, and where the globin disease mutation or the mutation in globin regulation causes production of a deleterious globin chain or production of insufficient quantities of a globin chain. The methods include transfecting the mammal with a vector comprising a mutant globin gene encoding the same globin chain as the deleterious of insufficient globin chain, where the vector is transfected under conditions where the mutant globin gene is expressed in the mammal, and where the mutant globin gene encodes a globin chain that has lower oxygen affinity and similar stability in hemoglobin when compared to the wild-type of the same globin chain. Also provided are methods of improving hemoglobin oxygen exchange in a mammal. The methods include transfecting the mammal with a vector comprising a mutant globin gene under conditions where the mutant globin gene is exp0ressed in the mammal, where the mutant globin gene encodes a globin chain that has lower oxygen affinity and similar stability in hemoglobin when compared to the same globin chain when unmutated.

### CROSS-REFERENCE TO RELATED APPLICATION

This application claims the benefit of U.S. Provisional Application No. 60/454,808, filed Mar. 14, 2003.

### BACKGROUND OF THE INVENTION

(1) Field of the Invention

The present invention generally relates to methods of treating hemoglobin diseases and insufficient oxygen exchange in mammals. More specifically, the invention relates to methods of improving oxygen exchange in mammals by transfecting the mammals with globin genes having lower affinity than wild-type globin genes.

(2) Description of the Related Art

### REFERENCES CITED

Adachi et al., 1996, Blood 87:1617-1624.

Huisman et al., 1996, A Syllabus of Human Hemoglobin Variants, l at http://globin.cse.psu.edu/html/huisman/variants/.

Imren et al., 2002, Proc. Natl. Acad. Sci. USA 99:14380-14385.

Pawliuk et al., 2001, Science 294:2368-2371.

Rao et al., 2000, J. Mol. Biol. 300:1389-1406.

Schneider et al., 1975, Biochim. Biophys. Acta 400:365-373.

Moo-Penn et al., 1981, Am. J. Hematol. 11:137-145.

U.S. Pat. No. 5,843,888.

U.S. Pat. No. 6,486,123.

Several important diseases are caused by mutant globin genes or the regulation of those genes. These include sickle cell disease, caused by a point mutation causing β^{E6V} (a commonly used designation indicating that the Glu [E in the universally accepted one letter code for amino acids] in wild-type β globin is substituted with Val [V]), and various thalassemias, where reduced, or no, α and/or β globin is made. Thalassemia can be due to a nonsense mutation in the globin gene, or mutations in regulatory elements of the globin gene.

Recent work with viral vectors have demonstrated that the deleterious effects of these globin diseases can be substantially ameliorated (Pawliuk et al., 2001; Imren et al., 2002).

The lentiviral vectors used in the above studies achieved high expression of the globin transgene, but did not reach the level of expression of normal globin chain synthesis. Thus, there remains a need to provide improvements in these methods in order to achieve hemoglobin function as close as possible to wild-type hemoglobin. The present invention addresses that need.

### SUMMARY OF THE INVENTION

Accordingly, the invention is based on the discovery that, in mammals with a disease caused by a globin disease mutation or a mutation in globin regulation, where the globin disease mutation or the mutation in globin regulation causes production of a deleterious globin chain or production of insufficient quantities of a globin chain, transfection of the mammal with a vector comprising a mutant globin gene encoding a globin chain that has lower oxygen affinity when compared to the same globin gene when unmutated, provides superior hemoglobin oxygen exchange in the mammal than transfection of the mammal with a vector comprising the unmutated gene.

Thus, in some embodiments, the invention is directed to methods of treating a mammal having a disease, the disease caused by a globin disease mutation or a mutation in globin regulation, where the globin disease mutation or the mutation in globin regulation causes production of a deleterious globin chain or production of insufficient quantities of a globin chain. The methods comprise transfecting the mammal with a vector comprising a mutant globin gene encoding the same globin chain as the deleterious or insufficient globin chain. In these methods, the vector is transfected under conditions where the mutant globin gene is expressed in the mammal, and the mutant globin gene encodes a globin chain that has lower oxygen affinity and similar stability in hemoglobin when compared to the same globin chain when unmutated.

In other embodiments, the invention is directed to methods of improving hemoglobin oxygen exchange in a mammal. The methods comprise transfecting the mammal with a vector comprising a mutant globin gene under conditions where the mutant globin gene is expressed in the mammal. The mutant globin gene in these methods encodes a globin chain that has lower oxygen affinity and similar stability in hemoglobin when compared to the same globin chain when unmutated.

Additionally, the invention is directed to vectors capable of stable transfection into a hematopoietic stem cell of a mammal. The vectors comprise a mutant globin gene, where the mutant globin gene encodes a globin chain having lower oxygen affinity and similar stability when compared to the same globin chain when unmutated. Mammalian cells, and nonhuman animals comprising any of these vectors is encompassed by the invention.

### DETAILED DESCRIPTION OF THE INVENTION

The present invention is based on the discovery that improved oxygen exchange can be achieved in tissues of mammals, including mammals having a disease caused by a globin disease mutation or a mutation in global regulation, by transfecting the mammal with a vector comprising a mutant globin gene encoding a globin chain that has lower oxygen affinity and similar stability in hemoglobin when compared to the same globin chain when unmutated.

Thus, in some embodiments, the invention is directed to methods of treating a mammal having a disease, the disease caused by a globin disease mutation or a mutation in globin regulation, where the globin disease mutation or the mutation in globin regulation causes production of a deleterious globin chain or production of insufficient quantities of a globin chain. The methods comprise transfecting the mammal with a vector comprising a mutant globin gene. In these methods, the vector is transfected under conditions where the mutant globin gene is expressed in the mammal, and the mutant globin gene encodes a globin chain that has lower oxygen affinity and similar stability in hemoglobin when compared to the same globin chain when unmutated. In preferred embodiments, the mutant globin chain encodes the same globin chain as the deleterious or insufficient globin chain. However, the chain does not necessarily have to encode the same globin chain as the deleterious or insufficient globin chain to be useful. See, e.g., Adachi et al., 1996, indicating that transfection with a y globin chain could be useful in treating sickle cell disease.

As used herein, “oxygen affinity” is the strength of binding of oxygen to a hemoglobin molecule. The P₅₀ (the oxygen pressure [e.g., measured in mm Hg] where the hemoglobin is 50% saturated) is a measure of oxygen affinity, and is measured by determination of the hemoglobin oxygen-dissociation curve. See, e.g., U.S. Pat. No. 6,486,123. A mutant globin chain has lower oxygen affinity, when compared to the same globin chain when unmutated, if hemoglobin made with the mutant globin chain has about 90% or less of the oxygen affinity of wild-type hemoglobin, i.e., has a P₅₀ value at least about 11% higher than wild-type hemoglobin at pH 7.4. Since wild-type hemoglobin has a P₅₀ value of about 10 mm Hg, a low affinity hemoglobin has a P₅₀ value of at least about 11 mm Hg. In preferred embodiments, the low affinity hemoglobin has an oxygen affinity of about 60% or less of wild-type hemoglobin.

“Stability” is a measure of the ability of a hemoglobin molecule to continue to function in oxygen exchange over time. As used herein, “stability” is inversely proportional to the autoxidation rate, measured as the autoxidation rate constant kₐᵤₜₒ(h⁻¹) (e.g., measured as described in U.S. Pat. No. 6,486,123). A mutant globin chain has similar stability when compared to the same globin chain when unmutated (i.e., wild-type) if hemoglobin made with the mutant globin chain has at least about 30% of the stability of wild-type hemoglobin, i.e., has an autoxidation rate constant less than about three times the autoxidation rate of the wild-type hemoglobin.

These methods can be used to treat any disease caused by a globin disease mutation or a mutation in globin regulation. As used herein, a globin disease mutation is a mutation in a normal globin gene that, when incorporated into a hemoglobin, a disease results. Non-limiting examples or diseases caused by globin disease mutations or mutations in globin regulation include thalassemia (α or β, including hemoglobin H disease and hydrops) and sickle cell anemia.

As used herein, a normal globin gene is a gene encoding a normal globin chain that, when present in hemoglobin, behaves similarly to the analogous wild-type globin chain when present in hemoglobin. A normal globin chain behaves similarly to the analogous wild-type globin chain when a hemoglobin incorporating the normal globin chain does not exhibit deleterious stability, oxygen affinity (i.e., higher than normal or very low [less than about 10% of wild-type]), or cooperativity, when compared to hemoglobin made with wild-type hemoglobin. Examples of amino acid sequences of wild-type human globin chains are provided herein as SEQ ID NO:1 (α globin), SEQ ID NO:2 (β globin), SEQ ID NO:3 (γ globin), SEQ ID NO:4 (δ globin), SEQ ID NO:5 (ε globin), and SEQ ID NO:6 (ζ globin). Sequences for wild-type globin chains for any other mammalian species are known or can be determined without undue experimentation. As is well known, there are many normal globin chains that are not wild-type chains. For many such human variants, see Huisman et al., 1996.

These methods are not limited to the use of any particular mutant globin gene encoding a globin chain that has lower oxygen affinity and similar stability in hemoglobin when compared to the same globin chain when unmutated. Several such mutant globin genes are known. Non-limiting examples include β^{D21G} (a β chain where the wild-type Asp at residue 21 is substituted with a Gly)(the mutant β chain incorporated in Hb Connecticut—Moo-Penn et al., 1981), β^{N108Q} (U.S. Pat. No. 6,486,123), β^{L105W} (Id.), α^{V96W} (U.S. Pat. No. 5,843,888), and α^{D94N} (Hb Titusville—Schneider et al., 1975).

The mutant globin gene conferring lower oxygen affinity can also include additional advantageous mutations, now known or later discovered, such as mutations that improve the stability or prevent a disease or other undesirable phenotype of the resulting hemoglobin. Non-limiting examples of such mutations include β^{T87Q}, which prevents polymerization of the abnormal sickle cell hemoglobin (HbS), and α^{L29F}, which improves stability (U.S. Pat. No. 6,486,123). The present invention also encompasses the use of two or more mutant globin genes conferring lower oxygen affinity and/or two or more mutations conferring lower oxygen affinity in a single globin gene.

These methods are useful for any mammal including nonhuman mammals such as rodents. Mice are the preferred nonhuman mammals, because the mouse model for hemoglobin transfection technology is well developed and has been established to be a standard model system for human hemoglobin transfection technology. The methods are also useful in primates including humans, e.g., to correct a human disease caused by a globin disease mutation or a mutation in global regulation.

The mutant globin gene can be from the same species or from a different species as the mammal. See, e.g., Rao et al., 2000, demonstrating that the interspecies hemoglobin hybrid of a pig α globin with the human β globin having the sickle cell mutation lacks the hemoglobin polymerization characteristic of sickle cell disease. However, it is preferred that the mutant globin gene be from the same species, to reduce the possibility of immune response to the extra-species globin chain.

The methods of the invention are not limited to the use of any specific vector, provided the vector allows expression of the lower oxygen affinity globin chain such that it is incorporated into the hemoglobin of the mammal. Examples of useful vectors include adenoviral vectors, naked DNA vectors, and, preferably, lentiviral vectors.

The vectors useful for the invention methods are also not limited to comprising any particular structure and can comprise any appropriate control elements, e.g., promoters, enhancers, etc, or other elements appropriate to the preferred expression of the mutant globin gene. A preferred vector comprises the mutant globin gene, a packaging signal, a central polypurine tract/DNA flap, a Rev-response element, a 3′ enhancer for the globin gene, and DNase 1 hypersensitive sites HS2, HS3 and HS4 of the large locus control region, all flanked by HIV long terminal repeats (Imren et al., 2002). When the mutant globin gene encodes a β chain, it is also preferred that the globin gene further comprises an IVS2 deletion. Such vectors are particularly useful for treating a mammal with β thalassemia (see Imren et al., 2002).

In these methods, transfection of the mammal with the vector can be achieved by any appropriate method known in the art. A preferred method is ex vivo transfection of hematopoietic stem cells with the vector, then transplantation of the cells into the mammal. The hematopoietic stem cells can be from the same mammal or from a second mammal.

In other embodiments of the invention, it is recognized that the mammal to be treated need not have a disease caused by a globin disease mutation or a mutation in globin regulation in order for the mammal to derive benefit from being transfected with the mutant globin genes. Since the mutant globin genes encode a globin chain that has lower oxygen affinity than the unmutated gene, animals having a disease characterized by reduced oxygen exchange, such as emphysema, would be expected to benefit from these methods. Indeed, it would be expected that the invention methods would cause improved hemoglobin oxygen exchange in healthy mammals.

Thus, the present invention is also directed to methods of improving hemoglobin oxygen exchange in a mammal. The methods comprise transfecting the mammal with a vector comprising a mutant globin gene under conditions where the mutant globin gene is expressed in the mammal. As with the previously described methods, the mutant globin gene encodes a globin chain that has lower oxygen affinity and similar stability in hemoglobin when compared to the wild-type of the same globin chain.

These methods are entirely analogous to the previously described methods, i.e., the mutant globin gene can comprise additional mutations, the methods can be utilized with any globin chain, the mutant globin gene can be any of those previously described as well as any other mutations causing the encoded globin chain to have lower oxygen affinity, and can be employed with any mammal.

The invention is also directed to vectors capable of stable transfection of a mutant globin gene into a hematopoietic stem cell of a mammal. In these embodiments, the vector comprises the mutant globin gene, where the mutant globin gene encodes a globin chain having lower oxygen affinity and similar stability when compared to the same globin chain when unmutated. The mutant globin gene in these vectors can encode any mutant globin chain, with α and β chains preferred.

These vectors are not limited to any specific type, provided the vector is capable of stable transfection into a mammalian cell, and allows expression of the lower oxygen affinity globin chain such that can be incorporated into the hemoglobin of the mammal. Examples include adenoviral, naked DNA, and, preferably, lentiviral vectors. A preferred vector comprises a packaging signal, a central polypurine tract/DNA flap, a Rev-response element, a 3′ enhancer for the globin gene, and DNase 1 hypersensitive sites HS2, HS3 and HS4 of the large locus control region, all flanked by HIV long terminal repeats.

The invention is additionally directed to mammalian cells transfected with any of the above vectors. These cells can be from any mammalian species, including rodents such as mice or rats, or from humans. The cells can be of any type, but are preferably hematopoietic stem cells.

The invention is also directed to non-human mammals transfected with any of the above vectors.

Other embodiments within the scope of the claims herein will be apparent to one skilled in the art from consideration of the specification or practice of the invention as disclosed herein. It is intended that the specification be considered exemplary only, with the scope and spirit of the invention being indicated by the claims which follow.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference in their entireties. The discussion of the references herein is intended merely to summarize the assertions made by the authors and no admission is made that any reference constitutes prior art. Applicants reserve the right to challenge the accuracy and pertinence of the cited references.

SEQ ID NO:s

Initial Met not counted
   SEQ ID NO:1—Human Wild-Type α Globin—from GenBank P01922.
0 mvlspadktn vkaawgkvga hageygaeal ermflsfptt ktyfphfdls hgsaqvkghg kkvadaltna
70 vahvddmpna lsalsdlhah klrvdpvnfk llshcllvtl aahlpaeftp
120 avhasldkfl asvstvltsk yr
   SEQ ID NO:2—Human Wild-Type β Globin—from GenBank NP000509.
0 mvhltpeeks avtalwgkvn vdevggealg rllvvypwtq rffesfgdls tpdavmgnpk
60 vkahgkkvlg afsdglahld nlkgtfatls elhcdklhvd penfrllgnv lvcvlahhfg
120 keftppvqaa yqkvvagvan alahkyh
   SEQ ID NO:3—Human Wild-Type γ Globin—from GenBank P02096.
0 mghfteedka titslwgkvn vedaggetig rllvvypwtq rffdsfgnls sasaimgnpk
60 vkahgkkvlt slgdaikhld dlkgtfaqls elhcdklhvd penfkllgnv lvtvlaihfg
120 keftpevqas wqkmvtavas alssryh
   SEQ ID NO:4—Human Wild-Type δ Globin—from GenBank P02042.
0 mvhltpeekt avnalwgkvn vdavggealg rllvvypwtq rffesfgdls spdavmgnpk
60 vkahgkkvlg afsdglahld nlkgtfsqls elhcdklhvd penfrllgnv lvcvlarnfg
120 keftpqmqaa yqkvvagvan alahkyh
   SEQ ID NO:5—Human Wild-Typeε Globin—from GenBank NP005321.
0 mvhftaeeka avtslwskmn veeaggealg rllvvypwtq rffdsfgnls spsailgnpk
60 vkahgkkvlt sfgdaiknmd nlkpafakls elhcdklhvd penfkllgnv mviilathfg
120 keftpevqaa wqklvsavai alahkyb
   SEQ ID NO:6—Human Wild-Type ζ Globin—from GenBank HZHU.
0 msltkterti ivsmwakist qadtigtetl erlflshpqt ktyfphfdlh pgsaqlrahg
60 skvvaavgda vksiddigga lsklselhay ilrvdpvnfk llshcllvtl aarfpadfta
120 eahaawdkfl svvssvltek yr

1-31. (canceled)

32. A method of improving hemoglobin oxygen exchange in a mammal, the method comprising transfecting the mammal with a vector comprising a mutant globin gene under conditions where the mutant globin gene is expressed in the mammal, wherein the mutant globin gene encodes a globin chain that has lower oxygen affinity and similar stability in hemoglobin when compared to the same globin chain when unmutated.

33. The method of claim 32, wherein the mutant globin gene further comprises at least one additional mutation.

34. The method of claim 32, wherein the globin chain is a β chain.

35. The method of claim 34, wherein the mutant globin gene encodes a globin chain that comprises a mutation selected from the group consisting of β^{D21G}, β^{N108Q}, and β^{L105W}.

36. The method of claim 35, wherein the mutation is β^{D21G}.

37. The method of claim 32, wherein the globin chain is an α chain.

38. The method of claim 37, wherein the mutant globin gene encodes a globin chain that comprises a mutation selected from the group consisting of α^{V96W} and α^{D94N}.

39. The method of claim 38, wherein the mutation is α^{D94N}.

40. The method of claim 32, wherein the mammal has a disease caused by a globin disease mutation or a mutation in globin regulation, where the globin disease mutation or the mutation in globin regulation causes production of a deleterious globin chain or production of insufficient quantities of a globin chain.

41. The method of claim 40, wherein the disease is a thalassemia.

42-46. (canceled)

47. The method of claim 40, wherein the mutant globin gene encodes a β chain and the disease is sickle cell anemia.

48. The method of claim 47, wherein the mutant globin gene encodes a globin chain that further comprises a β^{T87Q} mutation.

49. The method of claim 48, wherein the mutant globin gene encodes a globin chain that further comprises an α^{P114R} mutation.

50-54. (canceled)

55. The method of claim 32, wherein the vector is a lentivirus.

56. The method of claim 55, wherein the vector further comprises a packaging signal, a central polypurine tract/DNA flap, a Rev-response element, a 3′ enhancer for the globin gene, and DNase 1 hypersensitive sites HS2, HS3 and HS4 of the large locus control region, all flanked by HIV long terminal repeats.

57. The method of claim 56, wherein the mutant globin gene encodes a β chain and further comprises a IVS2 deletion.

58. The method of claim 57, wherein the disease is β thalassemia.

59. The method of claim 32, wherein the mammal is transfected by ex vivo transfection of hematopoietic stem cells with the vector, then transplantation of the cells into the mammal.

60. The method of claim 59, wherein the hematopoietic stem cells are from the same mammal.

61. The method of claim 59, wherein the hematopoietic stem cells are from a second mammal.
